(19) United States
(12) Patent Application Publication
(10) Pub. No.: US 2011/0046685 A1
(43) Pub. Date: Feb. 24, 2011

(54) SURGICAL INSTRUMENT FOR FITTING A KNEE PROSTHESIS

(76) Inventors: Eric FAURE, Laval (FR); Olivier ROCHETIN, Marcilly-Le-Chatel (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/843,553

(22) Filed: Jul. 26, 2010

(30) Foreign Application Priority Data
Jul. 24, 2009 (FR) 0955205

(51) Int. Cl. A61B 17/56

(52) U.S. Cl. 606/86 R

(57) ABSTRACT

Surgical instrumentation according to embodiments of the present invention may be configured to permit a surgeon to adjust external rotation of a femur according to two or more adjustment criteria selected from the group consisting of: (a) peroperative measurement of a value of the external rotation; (b) setting the external rotation to a preoperatively predetermined rotation value; (c) a position of a Whiteside line; (d) a position of a biepicondylar line; and (e) a position of an internal condyle.

### CROSS-REFERENCE TO RELATED APPLICATION

This application claims the foreign priority benefit of French Patent Application No. FR 0955205, filed on Jul. 24, 2009, which is incorporated herein by reference in its entirety for all purposes.

### TECHNICAL FIELD

The present invention relates to surgical instrumentation for preparing to fit a knee prosthesis, and more particularly to surgical instrumentation that gauges external rotation of the femur in two or more different ways.

### BACKGROUND

One often difficult aspect of positioning a femoral component of a knee prosthesis consists of selecting the appropriate anterior-posterior positioning of the femoral component with respect to what is commonly known as the external rotation of the femur with respect to the tibia of the patient undergoing surgery. This external rotation is with respect to the angular position of the femur about its longitudinal axis. Specifically, the fitting of a femoral prosthetic component entails, among other things, making anterior and posterior cuts in the femur, and dimensioning the cutting planes to form corresponding flat supports for attaching the femoral component. If the cutting planes are implemented without taking into consideration the external rotation of the femur, and in particular without taking the precaution of orienting the cutting planes with the same value of external rotation as the femur, or of making bone and/or ligament surgical corrections to adjust the value of the external rotation of the femur, the implanted femoral component may cause subluxation or luxation of the patient's patella.

In practice, there are a number of surgical techniques currently used for evaluating the external rotation of the femur and taking it into consideration when fitting the knee prosthesis. A surgeon often implements one of these various techniques according to the practices and the “schools” of the surgeons, and according to the clinical cases being treated, with each approach having its own unique set of instrumentation for implementation.

### SUMMARY

Embodiments of the present invention include instrumentation which, as far as the surgeons are concerned, makes it easier to take the external rotation of the femur into consideration when preparing to fit a knee prosthesis.

Embodiments of the present invention include a surgical instrument for preparing to fit a knee prosthesis, including a tibial support piece and a femoral support piece which are designed to press respectively against the upper end of the tibia and against the posterior condyles of the femur, so as to keep the tibia and the femur apart when the knee is flexed, a mounting and coupling mechanism for mounting and coupling the femoral support piece on the tibial support piece, this mounting and coupling mechanism configured so that when the tibial and femoral support pieces are pressing against the tibia and the femur, the external rotation of the femur can be adjusted selectively as a function of a criterion relating to a fitting technique chosen from among at least two criteria belonging to a group which includes: (a) peroperative measurement of the external rotation value, (b) preoperative setting to a predetermined rotation value, (c) the position of the Whiteside line, (d) the position of the biepicondylar line, and (e) the position of the internal condyle.

Embodiments of the present invention place at the surgeons' disposal a single piece of instrumentation with which the external rotation of the femur can be adjusted using one technique of at least two possible techniques, or in practice, of the five techniques that correspond to the five criterion in the group defined above. Using this single piece of surgical instrumentation, each surgeon will be able to select, even during the actual surgical intervention itself, which criterion to adopt for adjusting the external rotation of the femur. This adjustment is performed efficiently and accurately because it is performed while the knee joint is undergoing distraction under the action of the tibial support piece and of the femoral support piece. In practice, for this reason, the tibial support piece is used after the resection of the upper end of the tibia whereas the femoral support piece is brought into use following distal resection of the femur. Thus, having placed the tibia and the femur in relative tension, with the knee flexed to 90° beforehand to allow the support pieces to be set in position, the surgeon can visually assess the external rotation of the femur and measure it and/or, decide, as appropriate, whether to make bone and/or ligament surgical corrections in order to adjust this external rotation according to a criterion that the surgeon may choose, at its discretion, from the group of available criteria. Of course, the surgeon can adjust the external rotation of the femur successively according to two or more criteria from those of the group, by way of a verification or comparison.

Embodiments of the present invention may include additional features and/or characteristics, these being considered in isolation or in any technical feasible combination:

the mounting and coupling mechanism may include a piece mechanically interposed between the tibial support piece and the femoral support piece, with the interposed piece being mounted on the tibial support piece such that it can slide along an axis of sliding which extends in the direction of relative separation of the tibia and of the femur under the action of the tibial and femoral support pieces, whereas the femoral support piece is mounted on this interposed piece such that it can pivot about an axis of pivoting which is substantially perpendicular to the axis of the sliding and which, when the tibial and femoral support pieces are pressing against the tibia and the femur with the knee flexed, extends in the longitudinal direction of the femur;

the interposed piece is equipped with graduations which are distributed about the axis of pivoting and which are visually in register with a reference with which the femoral support piece is provided so that the value of the external rotation of the femur can be measured preoperatively; these graduations or other measure may be removably attached;

the mounting and coupling mechanism may include a reversible locking mechanism designed to immobilize the femoral support piece with respect to the interposed piece irrespective of the pivoted position of this femoral support piece about the axis of pivoting so as to fix the external rotation of the femur at a preoperative predetermined rotation value;

the interposed piece is provided with a spike which extends lengthways in a direction parallel to the axis of sliding and which, when the tibial and femoral support pieces are pressing against the tibia and the femur with the knee flexed, is visually in register with the distal side of the femur so that the value of the external rotation of the femur can be adjusted according to the position of the Whiteside line; this spike may be removably attached to the interposed piece;

the interposed piece is provided with a rod which extends lengthways in a direction perpendicular both to the axis of sliding and to the axis of pivoting and which, when the tibial and femoral support pieces are pressing against the tibia and the femur with the knee flexed, is visually in register with the distal side of the femur so that the external rotation of the femur can be adjusted according to the position of the biepicondylar line thereof; this rod or set of rods may be removably coupled with the interposed piece;

the mounting and coupling mechanism includes a set of shims, each of which is designed to be positioned and/or fixedly interposed between the femoral support piece and the external condyle of the femur and which have different respective thicknesses in the direction of this interposition so that the external rotation of the femur can be adjusted according to the position of the internal condyle;

the instrumentation further includes and/or supports a gauging piece for sensing or locating the anterior cortical bone of the femur, this gauging piece being configured to cooperate with the mounting and coupling mechanism, where the gauging piece may be used both to (a) choose from several femoral components of different respective anterio-posterior dimensions the femoral component to be fitted for this knee prosthesis and (b) to measure the distance, which could potentially be zero, between the anterio-posterior dimension of the chosen femoral component and a corresponding anterio-posterior dimension for the femur before it is fitted with the prosthesis;

the instrumentation may further include a mechanism configured to measure the relative separation between the tibia and the femur under the action of the tibial and femoral support pieces;

the tibial support piece may be configured to selectively support a first guide for positioning a femoral cutting block, the position of which is identified with respect to the femur by an anterior reference thereof, for example by feeling or locating the anterior cortical bone of the femur, and a second guide for positioning a femoral cutting block, the position of which is identified with respect to the femur by a posterior reference thereof, notably by resting against the femoral support piece with the interposition of the mounting and coupling mechanism;

the femoral support piece may be configured to be secured firmly to the femur, particularly on the distal side of the femur.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The invention will be better understood from reading the description which will follow, which is given solely by way of example and made with reference to the drawings in which:

FIGS. 1A, 1B and 1C illustrate a first operating phase, FIG. 1A being a perspective view of surgical instrumentation during use, while FIGS. 1B and 1C are elevation views along vertical planes IB and IC of FIG. 1A, respectively, according to embodiments of the present invention;

FIGS. 1D and 1E illustrate cross sections through the instrumentation of FIGS. 1A to 1C, with the instrumentation depicted on its own, the cross sections being taken along ID-ID and IE-IE of FIG. 1B, respectively;

FIG. 2 is a perspective view of the instrumentation illustrating a second operating phasem, according to embodiments of the present invention;

FIGS. 3A, 3B, 3C and 3D are perspective views of the surgical instrumentation respectively illustrating four possible ways in which to carry out a third operating phase;

FIGS. 4A and 4B illustrate a fourth operating phase, FIG. 4A being a perspective view of the surgical instrumentation, and FIG. 4B is a partial front elevation view this instrumentation, according to embodiments of the present invention;

FIG. 5 is a perspective view of the instrumentation, illustrating a fifth operating phase, according to embodiments of the present invention;

FIG. 6 illustrates a sixth operating phase, showing a femoral cutting block positioned using the instrumentation of the preceding figures, according to embodiments of the present invention; and

FIG. 7 is a view similar to FIG. 5, illustrating an alternative way of carrying out the fifth operating phase using the instrumentation, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

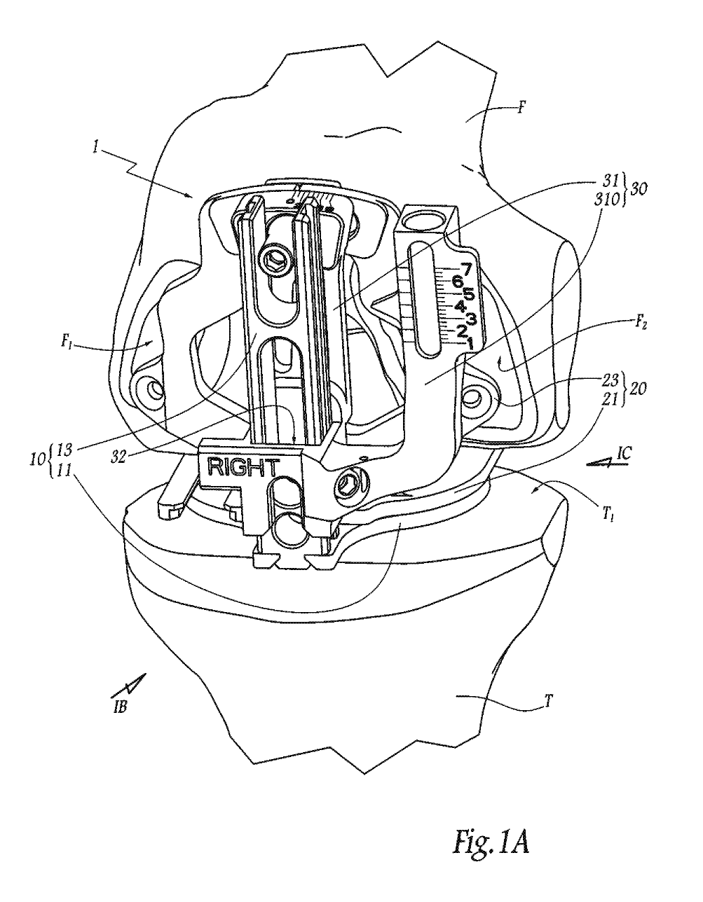

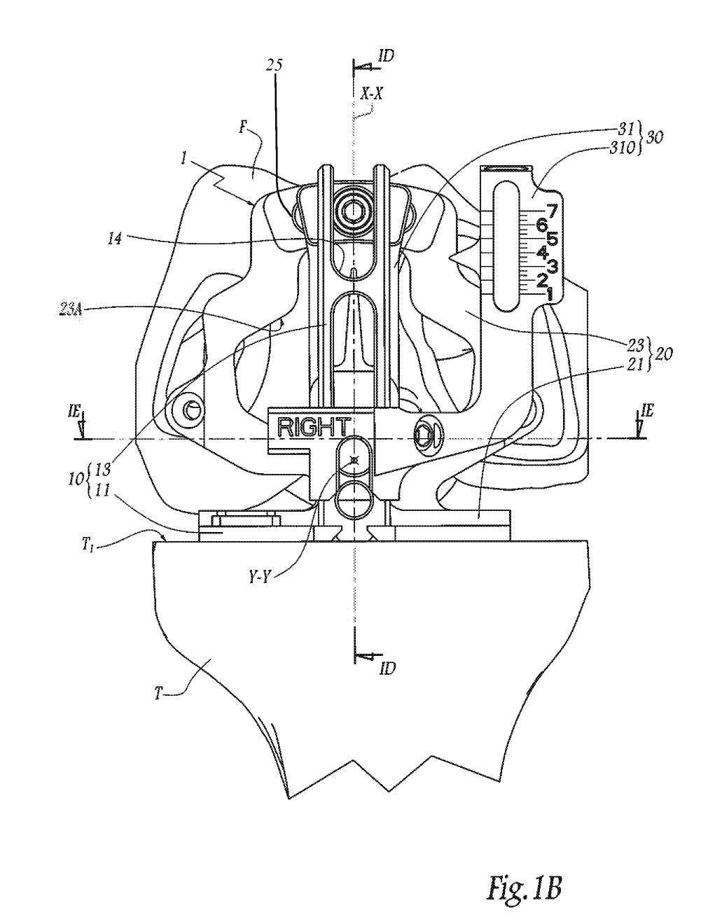

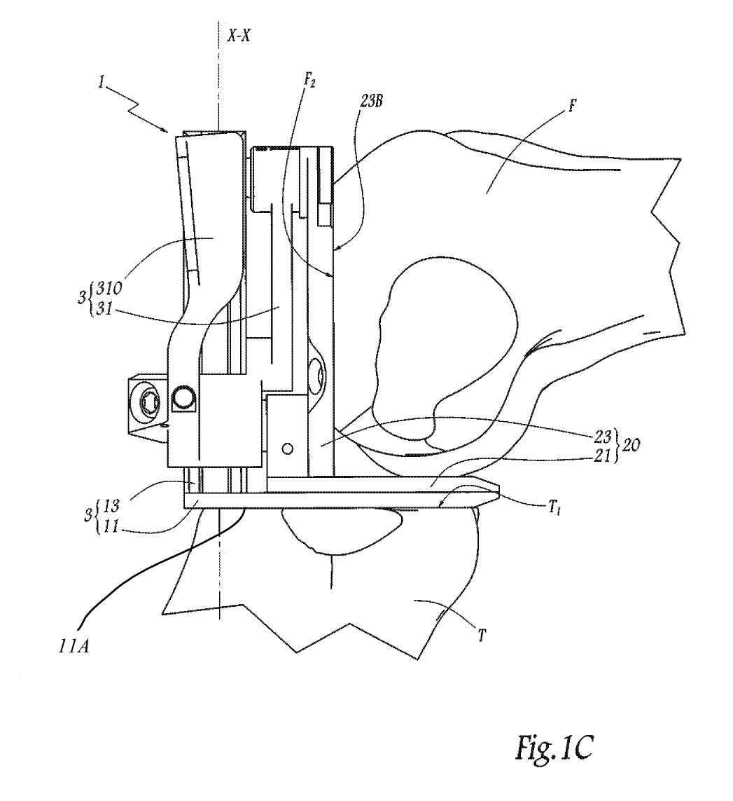

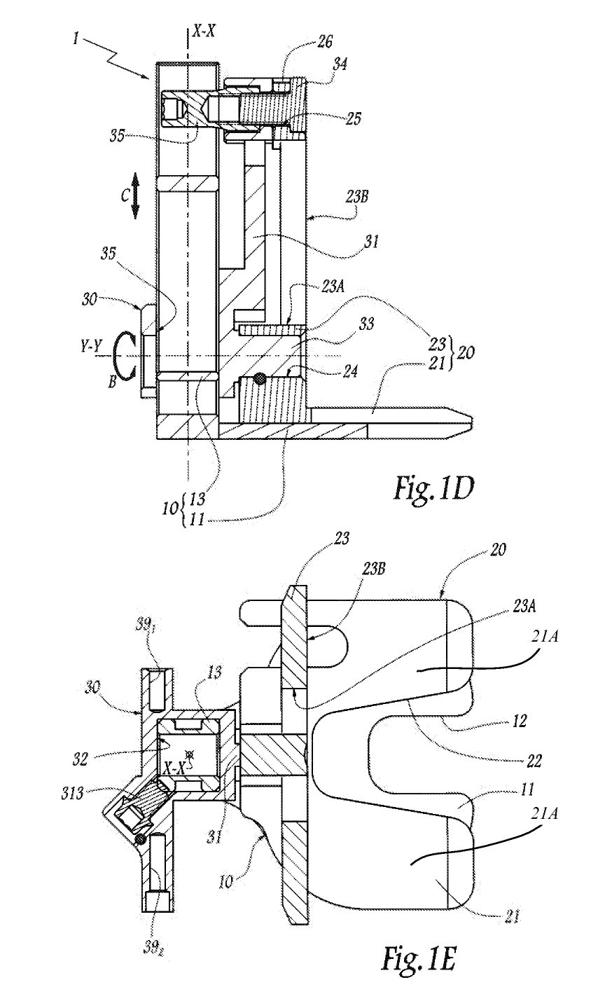

FIGS. 1 through 7 depict surgical instrumentation 1 including several components which will be detailed one after another during the course of the description of a use of this instrumentation for preparing to fit a knee prosthesis (not shown), between a tibia T and a femur F of a patient. This use of the instrumentation 1 will be set out in six operating phases, which may be performed in succession.

Moreover, throughout that which follows, the terms “upper”, “posterior”, “distal”, and the like, are to be understood in their anatomical sense, considering the patient who is undergoing surgery standing upright on a horizontal surface.

Prior to the first operating phase shown in FIGS. 1A to 1C, a surgeon makes an incision into the soft tissue surrounding the knee joint between the tibia T and the femur F in order to access the upper end of the tibia at the lower end of the femur. In a way known per se to one of ordinary skill in the art, the surgeon then resects, on the one hand, the upper end of the tibia, on a substantially horizontal plane of resection, so as thus to form a flat surface T₁ at the upper end of the tibia T and, on the other hand, the lower end of the femur F, on a plane of resection which is likewise horizontal when the patient's knee is extended, so as to form two distal flat surfaces F₁ and F₂ at the lower end of the femur F, these respectively being delimited by the external and internal distal condyles.

In order to proceed to the first operating phase, the surgeon then flexes the knee of the patient undergoing surgery, positioning the tibia T and the femur F at substantially 90° to one another, as shown in FIGS. 1A to 1C. The surgeon then sets in place, between the upper end of the tibia T and the lower end of the femur F, a preassembled collection of three components comprised by the surgical instrumentation 1, namely a tibial support piece 10, a femoral support piece 20, and a piece 30 interposed mechanically between the pieces 10 and 20. These three pieces 10, 20 and 30 are depicted on their own in FIGS. 1D and 1E.

Thus, the tibial support piece 10 comprises a rigid tibial plate 11 delimiting, on its face that is intended to face downward in service, a flat surface 11A adapted to press against the tibial surface T₁, forming a plane-plane contact as illustrated in FIGS. 1B and 1C. As illustrated in FIG. 1E, the tibial plate 11 has, in its posterior portion, a cutout 12 sized to accommodate the posterior knee ligaments, without damaging them, if these ligaments are kept. At its anterior end, the tibial plate 11 is rigidly provided with a post 13 which projects out, for example perpendicularly, from the face of the tibial plate 11 which is opposite to the flat surface 11A. This post 13 is centered on a longitudinal axis referenced X-X.

The femoral support piece 20 also comprises a rigid femoral plate 21 which, on its face intended to face upward in service, delimits two flat surfaces 21A which are separated from one another by a posterior cutout 22 of the femoral plate 21. These two flat surfaces 21A are designed to press against the posterior condyles of the femur F, the external and internal ones respectively, while the cutout 22 accommodates, without damaging them, the posterior knee ligaments if these are kept. At its anterior end, the femoral plate 21 is rigidly provided with a pivot mount 23 which projects out, for example perpendicularly, from the face of the femoral plate 21 that delimits the surfaces 21A. As illustrated in FIGS. 1B and 1D, this pivot mount 23 in its central region is provided with a through-hole 23A allowing surgical observations to be made through the pivot mount 23. On its posterior face, the pivot mount 23 delimits a flat surface 23B designed to press, forming plane-plane contact, against the two distal surfaces F₁ and F₂ of the femur F, as shown in FIG. 1C.

The piece 30 interposed between the tibial 10 and femoral 20 support pieces includes an elongate body 31 equipped, at one of its longitudinal ends intended to face downward during service, with a through-hole 32 designed to be fitted closely around the post 13 of the piece 10. Because of their complementing shape, the post 13 and the hole 32 together form a sliding connection sliding along the axis X-X as indicated by the double-headed arrow C in FIG. 1D. In addition, at the same end of the body 31 at which the hole 32 is provided, the body may also include a projecting peg 33 which extends from the posterior side of the body 31 in a direction perpendicular to the axis X-X. This peg 33, which externally is cylindrical on a circular base, being centered on an axis Y-Y perpendicular to the axis X-X, is housed as a close fit in a complementary orifice 24 delimited by the lower end part of the pivot mount 23 as illustrated in FIG. 1D. Collaboration between the orifice 24 and the peg 33 forms a pivoting connection with the pivoting being about the axis Y-Y, between the pieces 20 and 30, as indicated by the curved double-headed arrow B in FIG. 1D. At the same time, this arrangement connects the pieces 20 and 30 in terms of translational movement along the axis X-X with respect to the piece 10. The amplitude of the pivoting of the setup of pieces 20 and 30 is limited by a through-slot 25, delimited at the upper end of the pivot mount 23 with a curved longitudinal direction centered on the axis Y-Y. Passing right through this slot 25 is a peg 34 securely attached to the opposite end of the body 31 to the end at which the hole 32 and the peg 33 are provided. Advantageously, for reasons described below, the peg 34 is extended in a straight line by a protruding extension 35 which protrudes from the anterior face of the body 31 as shown in FIG. 1D.

Thus, during the first operating phase, the surgeon manipulates the pieces 10, 20 and 30 assembled with one another into a configuration in which the femoral plate 21 is pressed firmly against the tibial plate 11, sliding the piece 30 downward as far as it will go with respect to the piece 10. The plates 11 and 21 are designed to be pressed firmly together, forming plane-plane contact, such that the piece 20 then occupies a position termed a neutral position as far as its pivoting about the axis Y-Y is concerned. In practice, the pivoted position of the piece 20 with respect to the piece 30 is identified by a reference 26, illustrated here in the form of a groove formed in the upper end edge of the piece 20. Thus, in the neutral pivoted position, the reference 26 extends in a plane both perpendicular to the plates 11 and 21 and containing the axis X-X.

Having luxated the patient's patella, the surgeon introduces the plates 11 and 21 between the upper end of the tibia T and the lower end of the femur F until, on the one hand, the surface 11A is pressed firmly against the tibial flat surface T₁ and, on the other hand, the surface 23B is pressed firmly against the femoral distal surfaces F₁ and F₂, as shown in FIGS. 1A, 1B and 1C. The axes X-X and Y-Y are then positioned so that they are respectively perpendicular and parallel to the longitudinal direction of the femur.

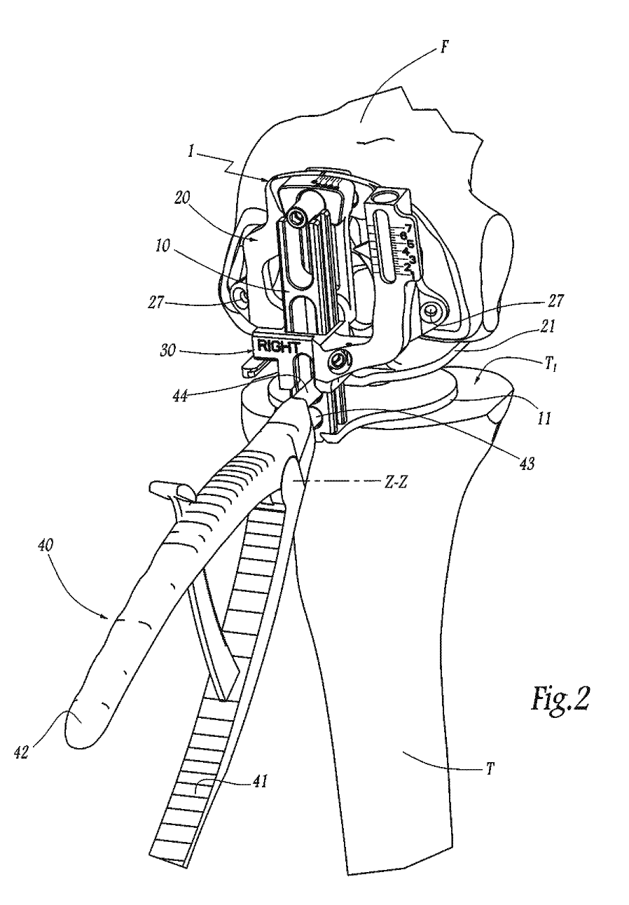

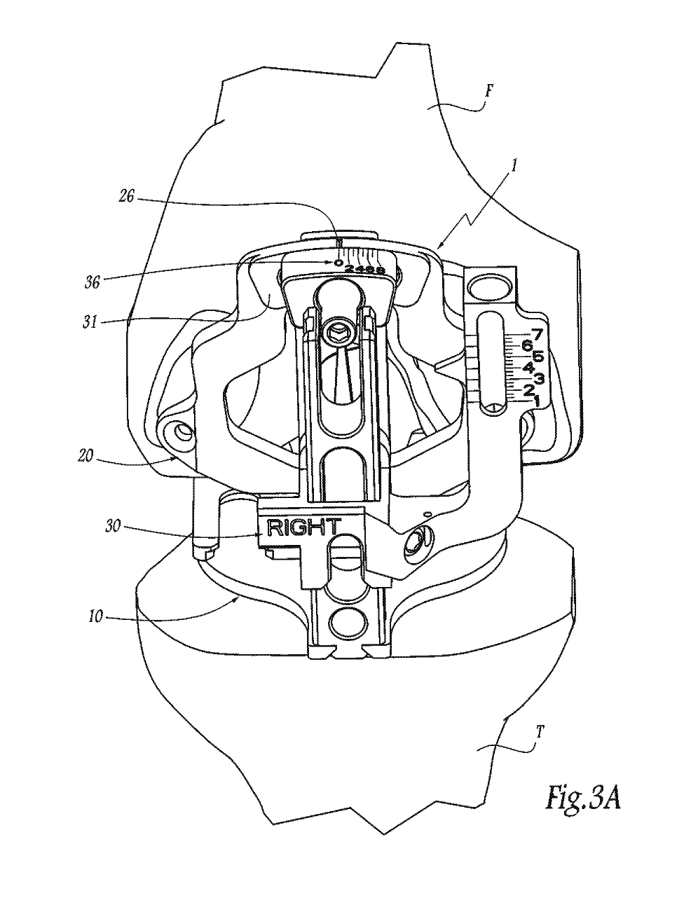

In a second operating phase shown in FIG. 2, the surgeon will distract or tension the knee joint undergoing surgery by separating the tibial 10 and femoral 20 support pieces from one another. To do this, in the exemplary embodiment illustrated, the surgeon uses distraction forceps 40 which comprise two branches 41 and 42 articulated to one another about an axis Z-Z which, in use, runs substantially perpendicular both to the axis X-X and to the axis Y-Y. Having connected the distal end 43 of the branch 41 to the tibial support piece 10 in terms of movement along at least the axis X-X, and after having connected the distal end 44 of the other branch 42 to the femoral support piece 20, via the interposed piece 30, in terms of movement along at least the axis X-X, the surgeon moves the respective proximal parts of the branches 41 and 42 relative to one another in such a way as to separate the distal ends 43 and 44. The result of this is that, with respect to the piece 10, the piece 30 is made to slide along the post 13 along the axis X-X and carries the piece 20 along with it in the same sliding movement. The path of this sliding is dictated by the collaboration between the hole 32 and the post 13 and, advantageously, by the collaboration between the extension 35 and a complementary groove 14, delimited by the free end of the post 13 and open at the top. The plates 11 and 21 therefore move apart in a direction of parting substantially parallel to the axis X-X, as shown in FIG. 2: the tibial plate 11 presses downward against the flat tibial surface T₁, whereas the femoral plate 21 presses upward against the posterior condyles of the femur F. Thus, the upper end of the tibia T and the lower end of the femur F move apart, in a vertical anatomical direction, tensioning the ligamento-muscular environment of the knee undergoing surgery.

Advantageously, before the tibia T and the femur F undergo distraction, the femoral support piece 20 can be fixed firmly to the femur, to stabilize the action on this bone. To do that, in the exemplary embodiment considered here, the plate 23 is provided, in each of its medial and lateral end parts, with a through-orifice 27, each orifice being configured to accept a bone anchor element (not depicted), such as but not limited to a screw, a rod, a pin, and the like.

The intensity of the tension applied between the tibia T and the femur F is either left to the discretion of the surgeon or controlled using a millimetric or dynamometric measurement tool. In any event, when their separation tension reaches an anatomically appropriate value, the branches 41 and 42 are locked off relative to one another, fixing the relative separation between the tibial 10 and femoral 20 support pieces.

Thus, on completion of the second operating phase, the femur F is connected in terms of movement to the femoral support piece 20 because of, on the one hand, the femoral plate 21 pressing against the posterior condyles of the femur under the action of the distraction forceps 40 and, on the other hand, the bone anchoring of attached elements passed through the holes 27.

In the third operating phase, the surgeon may adjust the external rotation of the femur F with respect to the tibia T. This external rotation of the femur corresponds to a rotational movement of the femur on itself, about the central longitudinal axis of the femur, under the action of the bone, ligament and muscle environment of the femur. Thus, when the tibia T and the femur F are placed under relative tension on completion of the second operating phase, the femur is liable to undergo spontaneous rotation, in practice by only a few degrees, which the surgeon will be able to adjust according to one criterion of a number of available criteria, using the instrumentation 1.

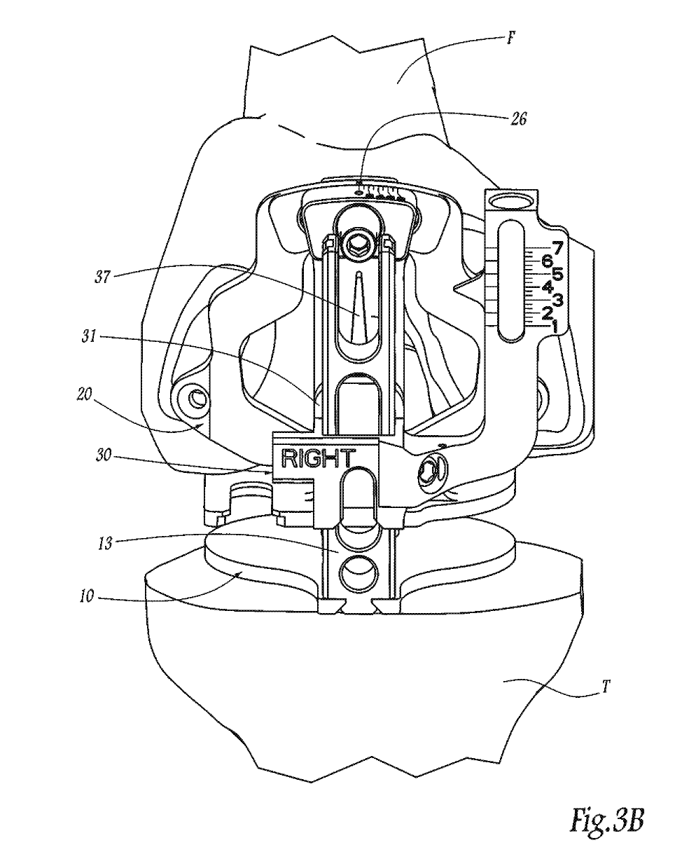

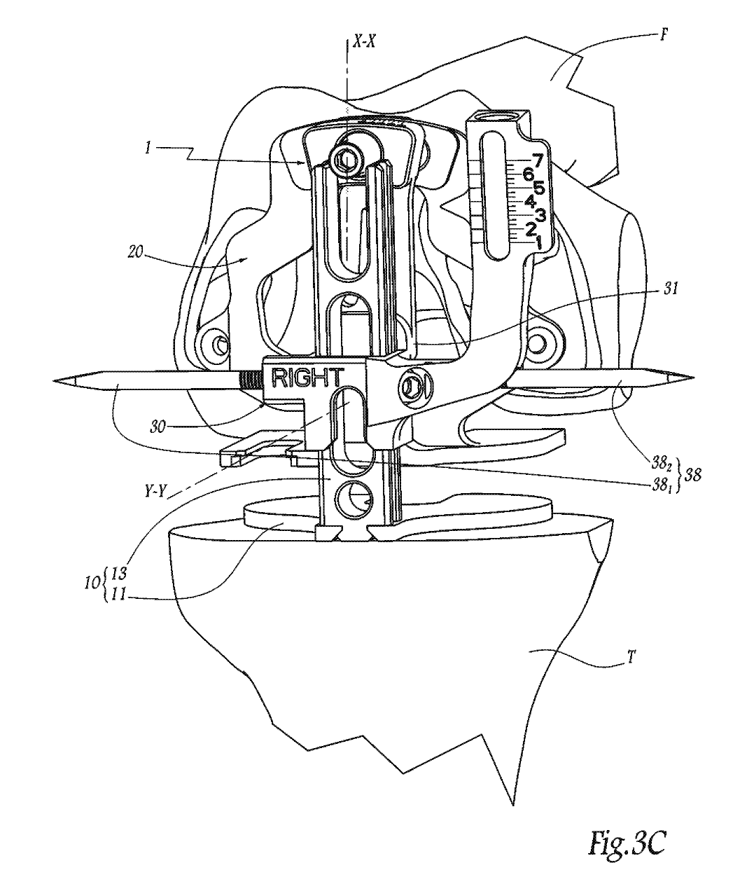

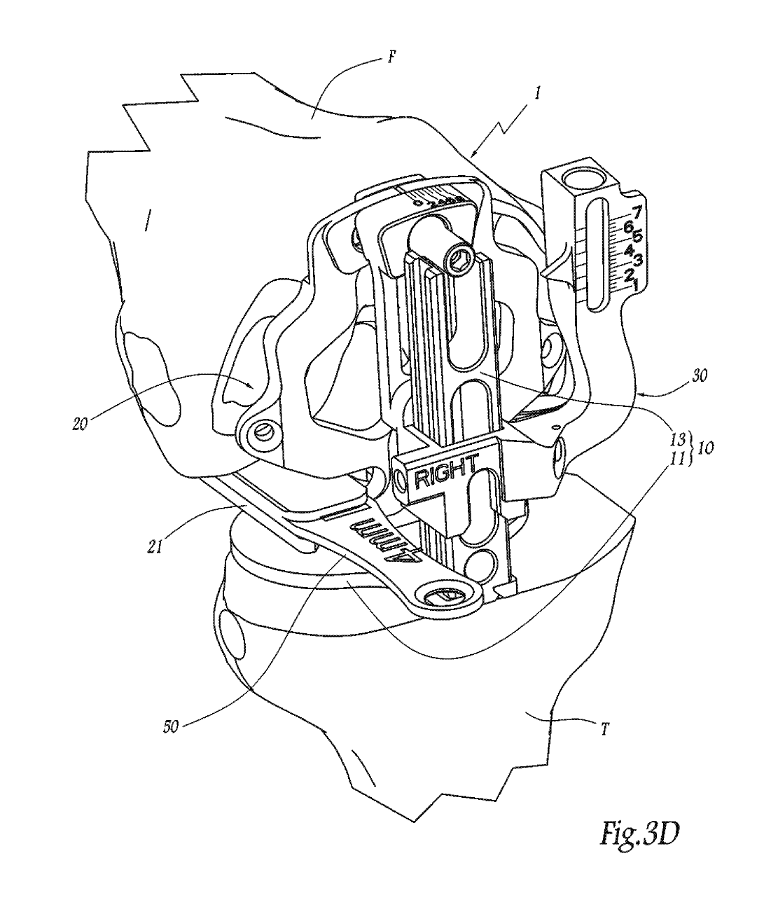

In order to illustrate this aspect of the instrumentation 1, the third operating phase mentioned hereinabove is illustrated by FIGS. 3A, 3B, 3C and 3D which illustrate various possible ways of adjusting this external rotation of the femur according to various criteria.

Thus, in FIG. 3A, the surgeon has the option of measuring the value of the external rotation of the femur F as a function of the ligament-muscular tension of the knee. To do this, the upper end face of the body 31 of the interposed piece 30 is provided with graduations 36, here in degrees or valuations ranging from “0” to “8”. In the exemplary embodiment illustrated in FIG. 3A, these graduations 36 are permanently borne by the piece 30 but, as an alternative, they could be attached removably. These graduations 36 may be distributed, in a straight line and/or radially, about the axis of pivoting Y-Y and are configured to be in visual register with the reference 26: by reading off from the graduations 36 which one is in register with the reference 26, the surgeon will known peroperatively the angular value of the pivoting of the femoral support piece 20 with respect to the interposed piece 30 about the axis Y-Y, and this gives the surgeon a measurement of the value of the external rotation of the femur F, because the femur F rotates in unison with the piece 20.

If the surgeon considers that the value of the external rotation of the femur is too great, that is to say too far removed from the neutral pivoting value which, in practice, corresponds to reference 26 being aligned with the graduation “0” of the graduations 36, the surgeon may make bone and/or ligament corrections in order to modify this value of rotation, until a peroperative measurement that conforms to the surgeon's expectations is achieved.

One undepicted alternative way of using the instrumentation 1 to adjust the external rotation of the femur F as a function of some criteria other than the peroperative measurement of this value of external rotation includes preoperatively setting a predetermined value for the external rotation of the femur. To do so, the surgeon, prior to the actual surgical intervention, determines this predetermined value on the basis of preoperative data supplied, for example, from radiology, scanner, and/or other medical images. Then, at the start of the third operating phase, the surgeon forces the femoral support piece 20 to pivot with respect to the interposed piece 30 about the axis Y-Y by an angle of pivoting that corresponds to the predetermined value of external rotation. To do this, the surgeon manipulates the femur F or the tibia T until the piece 20 reaches an angular position that suits his expectations. Next, the surgeon locks the piece 30 with respect to the piece 20 to fix the relative pivoting of these pieces. In this way, the next part of the surgical intervention will take place with the external rotation of the femur F set fixedly to the preoperative predetermined rotation value.

In the embodiment considered here, carrying out this alternative method comprises screwing the extension 35 about the peg 34 so as to grip and then reversibly lock the femoral support piece 20 against the upper end of the body 31 of the piece 30. This may be achieved by, for example, tightening the extension piece 35 to the peg 34 with their threadable engagement such that the upper end of the body 31 of piece 30 and the upper portion of the piece 20 are compressed between the peg 34 and the extension piece 35, according to embodiments of the present invention.

A third criterion on the basis of which the external rotation of the femur F can be adjusted is illustrated in FIG. 3B. This third criterion relates to what is commonly known as the Whiteside line which, in anatomy, corresponds to the anterio-posterior line drawn across the deepest part of the intercondylar notch of the femur. Thus, to adjust the external rotation of the femur, the surgeon visually compares the position of the Whiteside line against a spike 37 with which the interposed piece 30 is provided. Spike 37 may be rigidly or removably affixed to the interposed piece 30, according to embodiments of the present invention. This spike 37 extends along the body 31 in a direction parallel to the axis X-X, being situated across a through-opening which is delimited by the body 31 to allow anterio-posterior observations through body 31. Likewise, in order to allow the surgeon to observe the spike 37, the post 13 has an opening through it in an anterio-posterior direction, as shown in FIG. 3B.

FIG. 3C illustrates a fourth criterion against which the surgeon can adjust the external rotation of the femur F. Use of this criterion relies on the biepicondylar line, that is to say the line which, in anatomy, connects the extreme medial and lateral points of the condyles with the femur. In practice, the surgeon will visually compare the biepicondylar line with a rod 38 which is attached fixedly to the interposed piece 30. This rod 38 extends lengthwise in a direction perpendicular both to the axis X-X and to the axis Y-Y. In the exemplary embodiment illustrated here, the rod 38 is in fact made up of two separate rod parts 38₁ and 38₂ which are respectively attached to the medial and lateral sides of the body 31: one longitudinal end of each rod part 38₁, 38₂ is housed and secured, for example by screwing, in a complementary blind hole 39₁, 39₂ (FIG. 1E) delimited by the body 31. Thus, in use, the rod parts 38₁ and 38₂ embody the rod 38 which is visually in register with the distal side of the femur F, allowing the surgeon to assess whether or not this rod 38 is aligned with the biepicondylar line.

Finally, in FIG. 3D, the surgical instrumentation 1 comprises, in addition to the pieces 10, 20 and 30, a shim 50 interposed fixedly between the femoral plate 21 of the femoral support piece 20 and the external posterior condyle of the femur F. Given the thickness of the shim 50, in other words its dimension in the direction of the axis X-X, the presence of this shim alters the position of the external rotation of the femur. In fact, the external rotation of the femur is adjusted with respect to the internal condyle of which the position, with respect to the tibia, is fixed, being supported by the femoral plate 21.

In practice, the surgeon has at his disposal a set of several shims similar to the shim 50 but which have different respective thicknesses in the direction of the axis X-X: depending on which shim of the set is chosen and interposed between the external posterior condyle and the femoral plate 21, the external rotation of the femur can thus be adjusted with respect to a criterion connected to the position of the internal condyle.

Thus, on completion of the third operating phase, the surgeon has adjusted the external rotation of the femur F as a function of one criterion from the group including the aforementioned five criteria, namely the peroperative measurement of the value of this external rotation, the preoperative setting of a predetermined rotation value, the position of the Whiteside line, the position of the biepicondylar line and the position of the internal condyle. Any one of these five criteria, or even several of them successively, can be implemented using the one same piece of instrumentation 1, particularly using the pieces 10, 20 and 30 together with, if appropriate, the set of shims 50. This implementation may be performed with the tibia T and femur F separated from one another under the action of the distraction forceps 40, the distal ends 43 and 44 of which are respectively connected, in terms of movement, at least along the axis X-X, to the tibial 10 and femoral 20 support pieces.

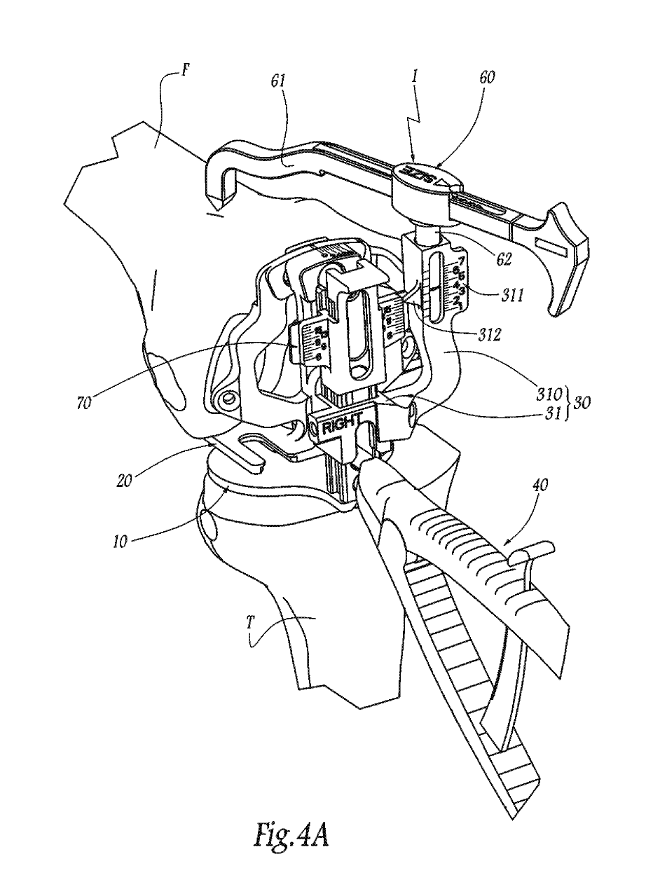

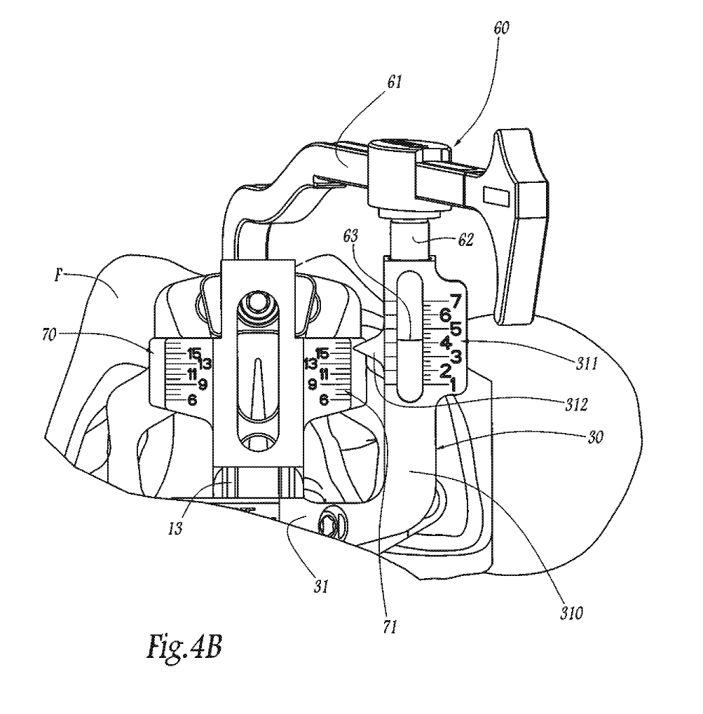

In a fourth operating phase, the surgeon may measure the anterio-posterior dimension of the lower end of the femur with a view to selecting, from several femoral components, the respective anterio-posterior dimensions of which differ, which femoral knee prosthesis component to fit. To do this, the surgeon uses a gauging piece 60 to feel for and/or locate the anterior cortical bone of the femur. As shown in FIGS. 4A and 4B, this piece 60 is designed to be attracted to the interposed piece 30. More specifically, the gauging piece 60 comprises an elongate arm 61 one of the ends of which is shaped so that it presses against the anterior cortical bone of the femur whereas, in its main span, the arm 61 is provided with a transverse finger 62 configured to be housed in a complementary fashion in a hollow shaft 310 secured to the body 31. This hollow shaft 310 runs parallel to the axis X-X and has, facing toward the surgeon, graduations 311, shown running from “1” to “7”, these respectively being associated with that same number of sizes of prosthetic femoral component. Thus, as shown in FIG. 4B, the surgeon visually compares a reference 63, here in the form of a line borne fixedly by the finger 62 of the gauging piece 60, against the graduations 311 to determine which size of prosthetic femoral component to select.

In the example depicted in FIG. 4B, the size of the prosthetic femoral component is assessed as falling between graduations “4” and “5”. Hence, if the surgeon chooses a femoral component of size “4”, the patient's knee, once the femoral and tibial components of the knee prosthesis have been implanted, will exhibit a certain laxity because, in flexing, the size of the prosthesis will be slightly smaller than the space available between the tibia and the femur, unless the surgeon implants a slightly thicker prosthetic tibial component. Conversely, if the surgeon chooses a femoral component of size “5”, the patient's knee, once the prosthesis has been implanted, will be tighter because the size of the prosthesis will slightly exceed the inter-bone space available, unless the surgeon releases the tension in the ligaments.

The graduations 311 make it possible to quantify the difference between the anterio-posterior dimension of the prosthetic femoral component chosen and the anterior posterior dimension of the femur before the prosthesis is fitted, using sub-graduations, notably millimetric graduations, between the graduations associated with implant sizes “1” to “7”. Thus, reconsidering the example shown in FIG. 4B, the difference using a size “4” is +1 mm, whereas the difference using a size “5” is −2 mm.

This quantification of the difference can then be correlated against a measurement of the inter-bone space available between the tibia T and the femur F when these bones are distracted or tensioned with respect to one another. To do this, the instrumentation 1 advantageously comprises a measuring piece 70 configured to be attached to the tibial support piece 10, here such that it slides on the post 13. This measuring piece 70 is dimensioned so that once it has been attached to the piece 10 it cooperates by visual correspondence with a reference 312 on the interposed piece 30, here situated along the hollow shaft 310. This reference 312 may be positioned facing graduations 71 with which the measuring piece 70 is provided, to make it possible to read off one value from among these graduations, that corresponds to the relative separation, along the axis X-X, of the tibia T and of the femur F under the action of the tibial 10 and femoral 20 support pieces created by the distraction forceps 40. Thus, in the example shown in FIG. 4B, the relative separation between the tibia and the femur is measured at 16 mm. This information allows the surgeon to choose the thickness of the prosthetic tibial component for later implantation.

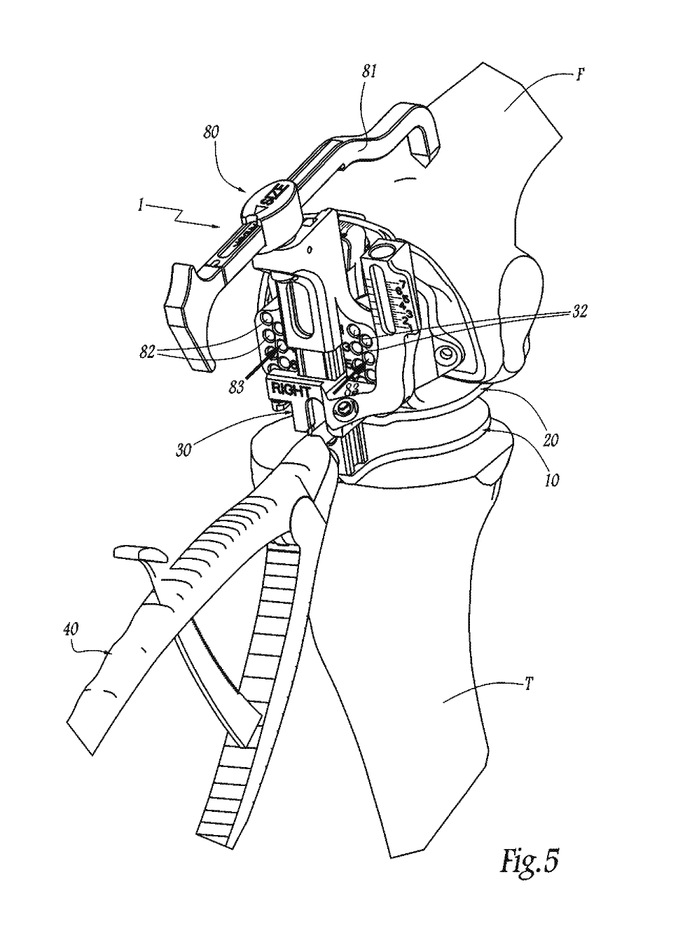

In a fifth operating phase illustrated in FIG. 5, the surgeon may prepare the positioning of a femoral cutting block to be used during the sixth operating phase described below. To do this, the surgeon uses a positioning guide 80 configured to be attached to the tibial support piece 10, which may be attached such that it can slide about the post 13. In the embodiment illustrated in FIG. 5, the positioning guide 80 includes a gauging arm 81 for feeling for and/or sensing the location of the anterior cortical bone of the femur F, which means that the femoral cutting block is positioned with respect to the anterior cortical bone of the femur. In other words, the cutting block is positioned, by the guide 80, using the anterior cortical bone of the femur as a point of reference. In addition, the positioning guide 80 is provided, on its distal and lateral sides respectively, with two series of seven through-holes 82, identified by the numerical references “1” to “7” respectively. In this way, the surgeon can identify, within each series of holes, which hole is associated with the numerical reference that corresponds to the size of the prosthetic femoral component chosen at the end of the fourth operating phase. By way of example, assuming that, at the end of the fourth operating phase, the surgeon selected size “4”, then the pair of holes 82 which are associated with the reference “4” would be employed for the insertion of two pins which are anchored into the distal side of the condyles of the femur, as indicated by the arrows 83 in FIG. 5. In practice, the holes 82 can be used, before the aforementioned pins are fitted, to guide a drill used to drill cavities in the femur to accept these pins.

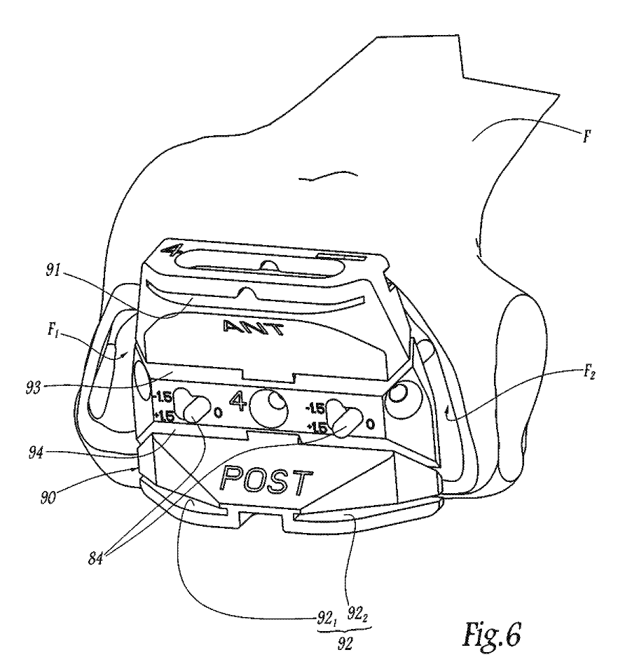

Finally, during a sixth operating phase illustrated in FIG. 6, the surgeon presses a femoral cutting block 90 against the distal side of the lower end of the femur F. Anterio-posterior positioning of this cutting block is achieved using the pins 84, fitted at the end of the fifth operating phase. In a way known to one of ordinary skill in the art, the cutting block 90 includes several cutting slots into each of which a saw blade is introduced to resect the lower end of the femur, in a plane flush with the slot. In particular, the cutting block 90 comprises an anterior cutting slot 91 and a posterior cutting slot 92 which may consist of a medial sub-slot 92₁ and a lateral sub-slot 92₂. The cutting block 90 may also include two further slots 93,94, facilitating creation of chamfers between the distal surfaces F₁ and F₂ of the femur, on the one hand, and the resection surface obtained using the anterior slot 91 and the resection surfaces obtained using the posterior sub-slots 92₁ and 92₂, on the other hand.

The cutting block 90 may be dimensioned so that, when positioned on the femur F using the pins 84, its anterior slot 91 and its posterior slot 92 make it possible to create femur resection surfaces which are positioned with respect to the femur with the anterior cortical bone of the femur as the point of reference. Of course, the anterio-posterior distance separating the slots 91 and 92 corresponds to the anterio-posterior size of the prosthetic femoral component to be implanted such that in practice the surgeon has a set of several cutting blocks each respectively associated with one of the components sizes “1” to “7”.

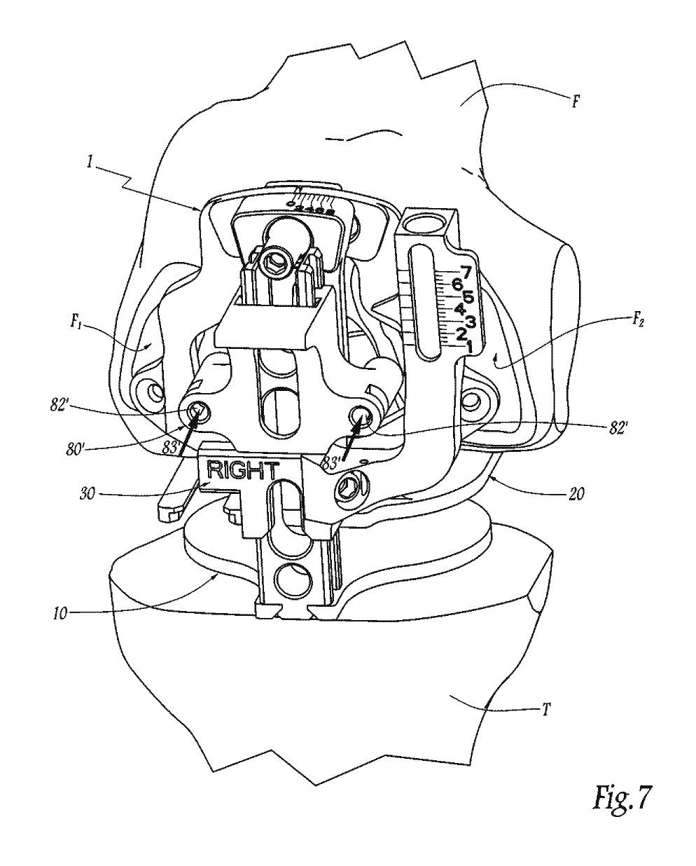

Rather than positioning the cutting blocks 90 using the anterior cortical bone of the femur as the point of reference for positioning, this being a first implementation option, a second optional way of using the instrumentation 1 is to position these cutting blocks using the posterior condyles of the femur as the point of reference. To do this, during the fifth operating phase, the surgeon uses not the positioning guide 80 but a positioning guide 80′ as shown in FIG. 7. This positioning guide 80′ is designed to be attached to the tibial support piece 10, in this instance such that it can slide about the post 13, until it presses along the X-X axis against the femoral support piece 20, with the interposition of the interposed piece 30. In this way, the positioning guide 80′ is positioned, with respect to the femur F, while at the same time being identifiable with respect to the posterior condyles of the femur, pressing against the femoral plate 21 of the piece 20. As a result, by dimensioning this guide 80′ appropriately, this guide allows two pins similar to the pins 84 to be fitted in to the distal surfaces F₁ and F₂ of the femur F, as indicated by the arrows 83′ in FIG. 7. These pins are then used, during the sixth operating phase, to set in place one of the cutting blocks 90, namely the one that corresponds to the adopted size of prosthetic femoral component. In this embodiment, unlike the positioning guide 80, the positioning guide 80′ has just one pair of holes 82′ into which fit the aforementioned pins and/or their corresponding drill bit: this is because provision has here been made for the anterio-posterior dimension of the various cutting blocks 90, between their posterior slots 92 and their pin holes, to be constant irrespective of the size of cutting block (whereas the anterio-posterior dimension between their pin holes and their anterior slots 91 varies according to the size of cutting block), according to embodiments of the present invention.

Thus, by virtue of the positioning guides 80 and 80′, the instrumentation 1 makes it possible, peroperatively, to choose the positioning reference, either anterior or posterior, for the cutting block 90 used in the sixth operating phase, according to embodiments of the present invention.

More generally, the surgical instrumentation 1 makes it possible to combine several operating techniques into a single set of preassembled components: for example, the tibial support piece 10, the femoral support piece 20, the interposed piece 30, and the distraction forceps 40. Furthermore, for each of these operating techniques, the instrumentation 1 allows the external rotation of the femur F with respect to the tibia T to be observed and/or adjusted as desired. Furthermore, the instrumentation 1 allows preparation to be carried out for fitting the knee prosthesis by being attached firmly and stably to the femur, without there being any need to remove and then refit some of the instrumentation. Also, the various components of the instrumentation 1 may be shaped in such a way as to give the surgeon good sight of the lower end of the femur F and of the upper end of the tibia T.

Various modifications and variations to the instrumentation 1 may be made. For example, the interposed piece 30 may optionally be locked in a chosen slide position, with respect to the tibial support piece 10; to do this, use may be made for example of a binding screw 313 (see FIG. 1E), which is screwed through the body 31 until it contacts and/or interferes with the post 13 in the hole 32; in this way, the flexing space between the tibia T and the femur F is arbitrarily fixed, advantageously being quantified accurately using the measuring piece 70, according to embodiments of the present invention.

Although the instrumentation 1 is illustrated being used on the right knee, a left knee can be treated using similar instrumentation including the same components as the instrumentation 1 except for the interposed piece 30 which is replaced by an interposed piece symmetric to the piece 30 in relation to a saggital plane of the patient, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. Surgical instrumentation configured to permit a surgeon to adjust external rotation of a femur according to two or more adjustment criteria selected from the group consisting of:
(a) peroperative measurement of a value of the external rotation;
(b) setting the external rotation to a preoperatively predetermined rotation value;
(c) a position of a Whiteside line,
(d) a position of a biepicondylar line; and
(e) a position of an internal condyle.

2. The surgical instrumentation of claim 1, wherein the surgical instrumentation is configured to permit the surgeon to adjust the external rotation of the femur according to three or more adjustment criteria selected from the group.

3. The surgical instrumentation of claim 2, wherein the surgical instrumentation is configured to permit the surgeon to adjust the external rotation of the femur according to four or more adjustment criteria selected from the group.

4. The surgical instrumentation of claim 3, wherein the surgical instrumentation is configured to permit the surgeon to adjust the external rotation of the femur according to each of the five adjustment criteria of the group.

5. Surgical instrumentation for preparing to fit a knee prosthesis, the surgical instrumentation comprising:
a tibial support element configured to press against a resected upper end of a tibia;
a femoral support element configured to press against posterior condyles of a femur, wherein the tibial support element and the femoral support element keep the tibia and the femur apart when the knee is flexed;
a mounting element rotatably coupled to one of the tibial support element and the femoral support element and slidably coupled to the other of the tibial support element and the femoral support element; and
a gauge configured to indicate rotation of the femoral support element when the femoral and tibial support elements are placed onto the knee and the femur is rotated about its longitudinal axis with respect to the tibia.

6. The surgical instrumentation of claim 5, wherein the mounting element is rotatably coupled to the femoral support element and slidably coupled to the tibial support element.

7. The surgical instrumentation of claim 5, wherein the gauge comprises graduation marks and a reference element, wherein the reference element moves with respect to the graduation marks according to the external rotation of the femur to permit measurement of a value of rotation peroperatively.

8. The surgical instrumentation of claim 6, further comprising a reversible locking mechanism configured to fix a position of the femoral support element with respect to the mounting element at any pivot position of the femoral support element with respect to the mounting element.

9. The surgical instrumentation of claim 8, wherein the reversible locking mechanism is configured to fix the position of the femoral support element with respect to the mounting element at a preoperatively predetermined rotation value.

10. The surgical instrumentation of claim 6, wherein the gauge comprises a spike extending substantially parallel to a direction of sliding between the mounting element and the tibial support element, wherein an angle formed between the spike and a Whiteside line of the knee when viewed from a front of the knee indicates the rotation.

11. The surgical instrumentation of claim 6, wherein the gauge comprises a rod extending substantially perpendicularly to both a direction of sliding between the mounting element and the tibial support element, and an axis of rotation of the mounting element with respect to the femoral support element, wherein an angle formed between the rod and a biepicondylar line of the knee when viewed from a front of the knee indicates the rotation.

12. The surgical instrumentation of claim 6, further comprising a set of shims, each of which is configured to be positioned between the femoral support element and an external condyle of the femur, wherein each shim of the set of shims has a different thickness, and wherein the external rotation of the femur can be adjusted according to the position of an internal condyle of the femur.

13. The surgical instrumentation of claim 6, further comprising a gauging piece configured to locate an anterior cortical bone of the femur, the gauging piece configured to cooperate with the mounting element to measure a distance between an anterio-posterior dimension of a certain femoral component and a corresponding anterio-posterior dimension of the femur, before the femur is fitted with the certain femoral component.

14. The surgical instrumentation of claim 6, wherein the gauge measures a relative separation between the tibia and the femur.

15. The surgical instrumentation of claim 6, further comprising:
a first guide mounted to the tibial support element, the first guide configured to position a femoral cutting block with reference to an anterior cortical bone of the femur, and
a second guide configured to position the femoral cutting block with reference to a posterior of the femur, by resting against the femoral support element with interposition of the mounting element.

16. Surgical instrumentation (1) for preparing to fit a knee prosthesis, comprising:
a tibial support piece (10) and a femoral support piece (20) which are adapted to press respectively against the upper end of the tibia (T) and against the posterior condyles of the femur (F), so as to keep the tibia and the femur apart when the knee is flexed,
mounting and coupling mechanism (30, 50) for mounting and coupling the femoral support piece on the tibial support piece, this mounting and coupling mechanism being configured so that when the tibial and femoral support pieces are pressing against the tibia and the femur, the external rotation of the femur can be adjusted selectively as a function of a criterion relating to a fitting technique chosen from among at least two criteria belonging to a group which includes peroperative measurement of the external rotation value, preoperative setting to a predetermined rotation value, the position of the Whiteside line, the position of the biepicondylar line, and the position of the internal condyle.

17. The instrumentation as claimed in claim 16, wherein the mounting and coupling mechanism comprises a piece (30) mechanically interposed between the tibial support piece (10) and the femoral support piece (20), the interposed piece being mounted on the tibial support piece such that it can slide along an axis of sliding (X-X) which extends in the direction of relative separation of the tibia and of the femur under the action of the tibial and femoral support pieces, whereas the femoral support piece is mounted on this interposed piece such that it can pivot about an axis of pivoting (Y-Y) which is substantially perpendicular to the axis of the sliding and which, when the tibial and femoral support pieces are pressing against the tibia and the femur with the knee flexed, extends in the longitudinal direction of the femur.

18. The instrumentation as claimed in claim 17, wherein the interposed piece (30) is equipped, with graduations (36) which are distributed about the axis of pivoting (Y-Y) and which are visually in register with a reference (26) with which the femoral support piece (20) is provided so that the value of the external rotation of the femur (F) can be measured peroperatively.

19. The instrumentation as claimed in claim 17, wherein the mounting and coupling mechanism comprises a reversible locking mechanism (34, 35) configured to immobilize the femoral support piece (20) with respect to the interposed piece (30) irrespective of the pivoted position of this femoral support piece about the axis of pivoting (Y-Y) so as to fix the external rotation of the femur (F) at a preoperative predetermined rotation value.

20. The instrumentation as claimed in claim 17, wherein the interposed piece (30) is provided with a spike (37) which extends lengthways in a direction parallel to the axis of sliding (X-X) and which, when the tibial (10) and femoral (20) support pieces are pressing against the tibia (T) and the femur (F) with the knee flexed, is visually in register with the distal side of the femur so that the value of the external rotation of the femur can be adjusted according to the position of the Whiteside line.

21. The instrumentation as claimed in claim 17, wherein the interposed piece (30) is provided with a rod (38) which extends lengthways in a direction perpendicular both to the axis of sliding (X-X) and to the axis of pivoting (Y-Y) and which, when the tibial (10) and femoral (20) support pieces are pressing against the tibia (T) and the femur (F) with the knee flexed, is visually in register with the distal side of the femur so that the external rotation of the femur can be adjusted according to the position of the biepicondylar line thereof.

22. The instrumentation as claimed in claim 17, wherein the mounting and coupling mechanism comprises a set of shims (50) each of which is designed to be positioned fixedly interposed between the femoral support piece (20) and the external condyle of the femur (F) and which have different respective thicknesses in the direction of this interposition so that the external rotation of the femur can be adjusted according to the position of the internal condyle.

23. The instrumentation as claimed in claim 17, and which further bears a gauging piece (60) for sensing the anterior cortical bone of the femur (F), this gauging piece being configured to cooperate with the mounting and coupling mechanism (30, 50), the gauging piece configured to be used to choose from several femoral components of different respective anterio-posterior dimensions the femoral component to be fitted for this knee prosthesis and to measure the distance between the anterio-posterior dimension of the chosen femoral component and a corresponding anterio-posterior dimension for the femur before it is fitted with the prosthesis.

24. The instrumentation as claimed in claim 17, and which further comprises means (70) for measuring the relative separation between the tibia (T) and the femur (F) under the action of the tibial (10) and femoral (20) support pieces.

25. The instrumentation as claimed in claim 17, wherein the tibial support piece (10) is configured to selectively support a first guide (80) for positioning a femoral cutting block (90), the position of which is identified with respect to the femur (F) by an anterior reference thereof, particularly by feeling the anterior cortical bone of the femur, and a second guide (80′) for positioning a femoral cutting block, the position of which is identified with respect to the femur by a posterior reference thereof, by resting against the femoral support piece (20) with the interposition of the mounting and coupling mechanism (30, 50).

26. A method for adjusting an external rotation of a femur at a knee, the method comprising:
resecting a top surface of a tibia to form a substantially flat tibial plane;
resecting an anterior surface of a femur to form a substantially flat femoral plane;
bending the knee until the substantially flat femoral plane is substantially perpendicular with the substantially flat tibial plane;
mounting a substantially flat surface of a tibial element against the substantially flat tibial plane;
mounting a substantially flat surface of a femoral element against the substantially flat femoral plane, and mounting the femoral element in contact with posterior condyles of the femur, wherein a mounting element is slidably coupled to one of the femoral element and the tibial element and rotatably coupled to the other of the femoral element and the tibial element;
separating the femur and the tibia by separating the femoral element and the tibial element;
rotating the femur substantially about its longitudinal axis; and
evaluating an external rotation of the femur with respect to the tibia by observing a gauge indicating the external rotation.

27. The method of claim 26, wherein the mounting element is rotatably coupled to the femoral support element and slidably coupled to the tibial support element.

28. The method of claim 26, wherein the gauge comprises graduation marks and a reference element, wherein the reference element moves with respect to the graduation marks according to the external rotation of the femur, and wherein evaluating the external rotation comprises peroperatively visually noting a position of the reference element with respect to the graduation marks.

29. The method of claim 27, further comprising:
preoperatively determining an external rotation value; and
reversibly locking the femoral support element with respect to the mounting element at a pivot position of the femoral support element with respect to the mounting element corresponding to the preoperatively determined external rotation value.

30. The method of claim 27, wherein the gauge comprises a spike extending substantially parallel to a direction of sliding between the mounting element and the tibial support element, wherein evaluating the external rotation comprises peroperatively visually noting a position of the spike with respect to a Whiteside line of the knee when viewed from a front of the knee.

31. The method of claim 27, wherein the gauge comprises a rod extending substantially perpendicularly to both a direction of sliding between the mounting element and the tibial support element, and an axis of rotation of the mounting element with respect to the femoral support element, wherein evaluating the external rotation comprises peroperatively visually noting a position of the rod with respect to a biepicondylar line of the knee when viewed from a front of the knee.

32. The method of claim 27, further comprising selecting a shim from a set of shims, each of the shims being configured to be positioned between the femoral support element and an external condyle of the femur, wherein each shim of the set of shims has a different thickness, and wherein evaluating the external rotation of the femur comprises visually noting a position of an internal condyle of the femur when the shim is positioned between the femoral support element and the external condyle.

33. The method of claim 27, further comprising using a gauging piece to locate an anterior cortical bone of the femur, the gauging piece configured to cooperate with the mounting element to measure a distance between an anterio-posterior dimension of a certain femoral component and a corresponding anterio-posterior dimension of the femur, before the femur is fitted with the certain femoral component.
